(19) United States
(12) Patent Application Publication
(10) Pub. No.: US 2006/0173068 A1
(43) Pub. Date: Aug. 3, 2006

(54) Amorphous and crystalline forms of dorzolamide hydrochloride and processes of making same

(76) Inventors: Judith Aronhime, Rehovot (IL); Csilla Nemethne Racz, Tiszavasvari (HU); Adrienne Kovacsne-Mezei, Debrecen (HU); Csaba Szabo, Debrecen (HU); Laszlo Zsolt Kovacs, Debrecen (HU); Lorant Gyuricza, Nyekljdhiza (HU)

Correspondence Address: KENYON & KENYON LLP, ONE BROADWAY, NEW YORK, NY, 10004, US

(21) Appl. No.: 11/334,991

(22) Filed: Jan. 18, 2006

Related U.S. Application Data
(60) Provisional application No. 60/645,363, filed on Jan. 18, 2005.
(60) Provisional application No. 60/664,141, filed on Mar. 21, 2005.
(60) Provisional application No. 60/700,507, filed on Jul. 18, 2005.

(51) Int. Cl. A61K 31/382; C07D 495/02

(52) U.S. Cl. 514/432; 549/23

(57) ABSTRACT

The invention provides an amorphous form of Dorzolamide hydrochloride, processes for the preparation of amorphous Dorzolamide hydrochloride, processes for making Form II from amorphous Dorzolamide HCl, a crystalline form of Dorzolamide hydrochloride, Form IV, process for making Form IV, and pharmaceutical compositions of amorphous Dorzolamide hydrochloride, Dorzolamide hydrochloride Form II and Dorzolamide hydrochloride Form IV.

### RELATED APPLICATIONS

This application claims the benefits of U.S. Provisional Patent Application No. 60/645,363, filed Jan. 18, 2005, U.S. Provisional Patent Application No. 60/664,141, filed Mar. 21, 2005, and U.S. Provisional Patent Application No. 60/700,507, filed Jul. 18, 2005, the contents of which are incorporated herein by reference in their entirety.

### FIELD OF THE INVENTION

The present invention is directed to amorphous Dorzolamide hydrochloride, to Dorzolamide hydrochloride form IV, to processes for their preparations, and to pharmaceutical compositions comprising them. This invention further relates to a novel process for preparing crystalline Dorzolamide hydrochloride Form II.

### BACKGROUND

Dorzolamide hydrochloride has the structure of Formula I. 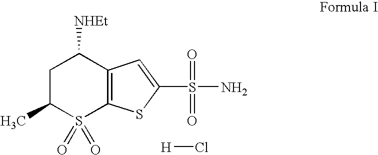
Dorzolamide hydrochloride, known chemically as 5,6-dihydro-4-(S)-ethylamino-6-(S)-methyl-4H-thieno-[2,3-b]thiopyran-2-sulfonamide-7,7-dioxyde hydrochloride, is a topically effective carbonic anhydrase inhibitor useful in the treatment of ocular hypertension. The Dorzolamide hydrochloride is marketed under the name “Cosopt” and “Trusopt” which is prescribed as eye-drops, and is formulated for topical ophthalmic use. Trusopt is supplied as Trusopt 2%. Cosopt is supplied as Dorzolamide hydrochloride in a combination with Timolol maleate.

The initial recommended dose is one drop in the affected eye(s) three times daily (Trusopt), or one drop in the affected eye(s) two times daily (Cosopt).”

ANALYTICAL PROFILES OF DRUG SUBSTANCES AND EXCIPIENTS, volume 26, pages 283-371, 1999, Academic Press, discloses two crystalline forms of Dorzolamide hydrochloride, which are identified as Form I and Form II. A third polymorphic form, Form III, is reported in International Patent Application No. WO 2004/089957. Also, WO 2004/016620 reports an anhydrous Form A, which is equivalent to Form II, and a monohydrate Form B.

The present invention relates to the solid state physical properties of Dorzolamide hydrochloride. These properties can be influenced by controlling the conditions under which Dorzolamide hydrochloride is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

The present invention also relates to solvate of Dorzolamide hydrochloride. When a substance crystallizes out of solution, it may trap molecules of solvent at regular intervals in the crystal lattice. Solvation also affects utilitarian physical properties of the solid state like flowability and dissolution rate.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

There is a need in the art for new polymorphs of Dorzolamide hydrochloride and processes for the preparation of Dorzolamide hydrochloride forms.

### SUMMARY OF THE INVENTION

In one embodiment, the present invention is directed to an amorphous form of Dorzolamide hydrochloride.

In another embodiment, the present invention is directed an amorphous form of dorzolamide that does not spontaneously convert to any crystalline form, at a temperature of about 40° C. or less, at a relative humidity of less than about 40 percent, for a period of time of at least about 3 months.

In another embodiment, the present invention provides a process for preparing amorphous form of dorzolamide hydrochloride by spray-drying a solution of Dorzolamide hydrochloride in a solvent selected from the group consisting of water, methanol, ethanol and mixtures thereof.

In another embodiment, the present invention provides a process for preparing Dorzolamide HCl Form II, comprising exposing amorphous Dorzolamide HCl to a relative humidity of more than about 40 percent, and recovering Dorzolamide HCl Form II.

In one embodiment, the present invention is directed to a crystalline form of Dorzolamide hydrochloride, designated as Form IV characterized by at least one of an X-ray powder diffraction pattern, having peaks at about 8.5°, 11.4°, 15.9°, and 18.7°±0.2° 2θ, an FT-IR spectrum, spectrum, having peaks at about 3275, 1620, 1305 and 1144 cm⁻¹, and a DSC thermogram, having two endothermic peaks at about 174° C. and in the range of about 278° to about 281° C.

In another embodiment, the present invention provides a process for preparing Dorzolamide HCl Form IV by precipitating it from a solution comprising Dorzolamide hydrochloride and N,N-dimethylacetamide. The process comprises dissolving Dorzolamide hydrochloride in N,N-dimethylacetamide, cooling the solution until a slurry is obtained, and recovering the crystalline form.

The present invention comprises pharmaceutical composition comprising Dorzolamide hydrochloride form IV or amorphous form and at least one pharmaceutically acceptable excipient.

The present invention comprises pharmaceutical composition comprising Dorzolamide hydrochloride of any of the forms II, IV or the amorphous form made by the processes of the invention, and at least one pharmaceutically acceptable excipient.

The present invention further encompasses a process for preparing a pharmaceutical formulation comprising combining the Dorzolamide hydrochloride form IV or amorphous form with at least one pharmaceutically acceptable excipient.

The present invention further encompasses a process for preparing a pharmaceutical formulation comprising combining the Dorzolamide hydrochloride of any of the forms II, IV or the amorphous form made by the processes of the invention, with at least one pharmaceutically acceptable excipient.

The present invention further encompasses the use of dorzolamide hydrochloride form IV or amorphous form for the manufacture of a pharmaceutical composition.

The present invention further encompasses the use of dorzolamide hydrochloride of any of the forms II, IV or the amorphous form made by the processes of the invention, for the manufacture of a pharmaceutical composition.

### BRIEF DESCRIPTION OF THE DRAWINGS

FIG. 1 illustrates a characteristic X-ray powder diffraction pattern of the amorphous form of dorzolamide HCl;

FIG. 2 illustrates a characteristic DSC curve of the amorphous form of dorzolamide HCl;

FIG. 3 illustrates a characteristic TGA curve of the amorphous form of dorzolamide HCl;

FIG. 4 is a non-polarized light micrograph of amorphous Dorzolamide HCl crystals;

FIG. 5 illustrates X ray diffraction patterns of an initially amorphous Dorzolamide HCl sample following 5 day exposure to relative humidity ranging from 0 to 100 percent;

FIG. 6 illustrates X ray diffraction of an initially amorphous Dorzolamide HCl before and after heating;

FIG. 7 illustrates a characteristic X-ray powder diffraction pattern of Dorzolamide hydrochloride crystal Form IV;

FIG. 8 illustrates a characteristic FT-IR spectrum of Dorzolamide hydrochloride crystal Form IV;

FIG. 9 illustrates a characteristic DSC curve of Dorzolamide hydrochloride crystal Form IV; and

FIG. 10 illustrates a characteristic TGA thermogram spectrum of Dorzolamide hydrochloride crystal Form IV;

FIG. 11 is a non-polarized light micrograph of Dorzolamide HCl Form IV crystals; and

FIG. 12 illustrates an equilibrium water content of Dorzolamide HCl amorphous at different relative humidity.

### DETAILED DESCRIPTION OF THE INVENTION

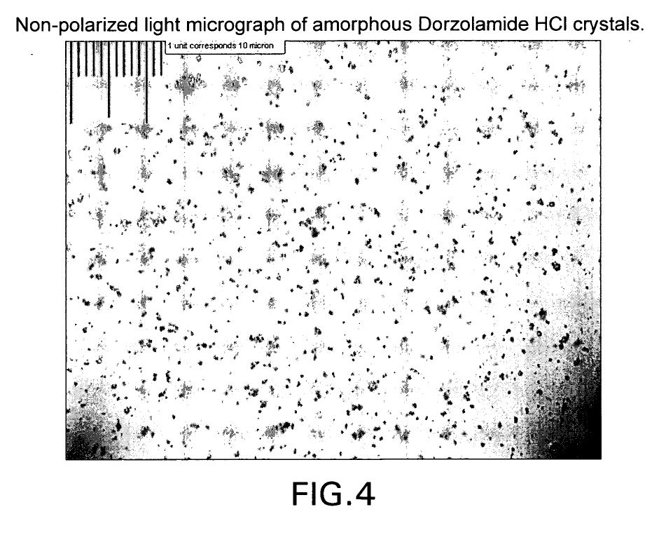

In one embodiment, the present invention is directed to an amorphous form of Dorzolamide hydrochloride. The amorphous form of Dorzolamide hydrochloride has an x-ray powder diffraction pattern that shows that the amorphous Dorzolamide HCl is free of XRD peaks from any crystalline form. The amorphous form of Dorzolamide hydrochloride may be further characterized by a DSC thermogram with an exothermic peak at about 130° C. to about 180° C. and an endothermic peak at about 284° C. The amorphous form of Dorzolamide hydrochloride may be further characterized by a TGA thermogram showing a weight loss of about of about 3 percent up to 170° C. and a sharp weight loss at about 230° C. The TGA with the weight loss of about 3 percent up to 170° C. corresponds to the result of the Karl Fischer titration. Appropriate PXRD, DSC and TGA figures correspond to figure numbers 1, 2 and 3, respectively. The amorphous form of Dorzolamide hydrochloride may be further characterized by a microscopic analysis that demonstrates that the material comprises irregularly shaped particles having a size of up to about 10 to about 20 μm. A non-polarized light micrograph of particles of amorphous Dorzolamide HCl is provided in FIG. 4. Preferably, the amorphous form of dorzolamide hydrochloride contains less than about 10%, more preferably, less than about 5%, more preferably less than about 2%, more preferably, less than about 1%, most preferably, less than about 0.2%, of any crystalline form of dorzolamide hydrochloride.

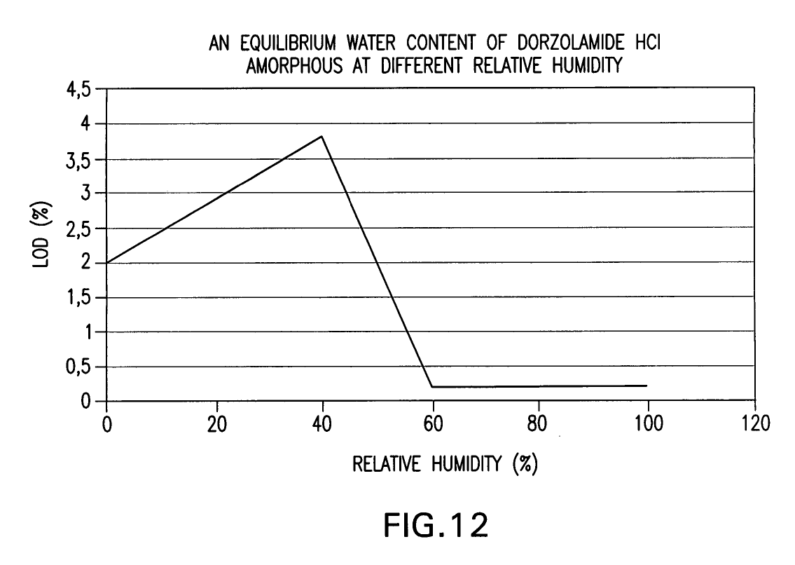

In another embodiment, the present invention is directed to an amorphous form of dorzolamide that does not spontaneously convert to any crystalline form, at a temperature of about 40° C. or less, at a relative humidity of less than about 40 percent, for less than about 2 months, as depicted in table 1. The results in this table are presented in FIG. 12.

TABLE 1
LOD Values (%) and Crystal Form of Samples of Amorphous
Dorzolamide HCl Stored at Different Relative Humidities

| RH (%) | LOD (%) | Crystal form by XRD |
|---|---|---|
| Original | 2.7 | amorphous |
| 0 | 2.0 | amorphous |
| 20 | 2.9 | amorphous |
| 40 | 3.8 | amorphous |
| 60 | 0.2 | Form II |
| 80 | 0.2 | Form II |
| 100 | 0.2 | Form II |

In another embodiment, the present invention provides a process for preparing amorphous form of dorzolamide hydrochloride by spray-drying, comprising the steps of dissolving Dorzolamide hydrochloride in a solvent selected from the group consisting of water, methanol, ethanol and mixtures thereof, and spray drying the solution to obtain amorphous Dorzolamide hydrochloride.

Spray drying broadly refers to processes involving breaking up liquid mixtures into small droplets (atomization) and rapidly removing solvent from the mixture. In a typical spray drying apparatus, a strong driving force evaporates the solvent from the droplets, which may be provided by providing a drying gas. Spray drying processes and equipment are described in Perry's Chemical Engineer's Handbook, pgs. 20-54 to 20-57 (Sixth Edition 1984).

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream from the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. The process of the invention is not limited to the use of such drying apparatuses as described above.

Spray drying may be performed in a conventional manner in the processes of the present invention (see, e.g., Remington: The Science and Practice of Pharmacy, 19th Ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The Dorzolamide hydrochloride product produced by spray drying may be recovered by techniques commonly used in the art, such as using a cyclone or a filter.

Preferably, the solution has a Dorzolamide hydrochloride concentration of about 10 to about 100 g/l. Preferably, the solution is stirred. Preferably, the solution is stirred for about 10 to about 60 minutes. Preferably, the solution is filtered, preferably, through a G3 or G5 sintered glass filter using vacuum. Optionally, the filtered solution is then diluted with water to provide a dorzolamide solution having a concentration of from about 0.5 to about 10 m/m percent. Preferably, the solution is dried at an inlet temperature of about 110° C. to about 130° C., more preferably, at an inlet temperature of about 120° C. to about 125° C. Preferably, the solution is dried at an outlet temperature of about 40° C. to about 60° C., more preferably, at an outlet temperature of about 53° to about 60° C. Preferably, the air speed during the spray-drying is of about 600 liters per hour. Preferably, the feed rate of the solution during the spray-drying is of about 225 to about 375 ml per hour, for about 90 minutes. Preferably, the drying process produces a yield of at least about 50 percent by weight, based on the initial weight of Dorzolamide hydrochloride.

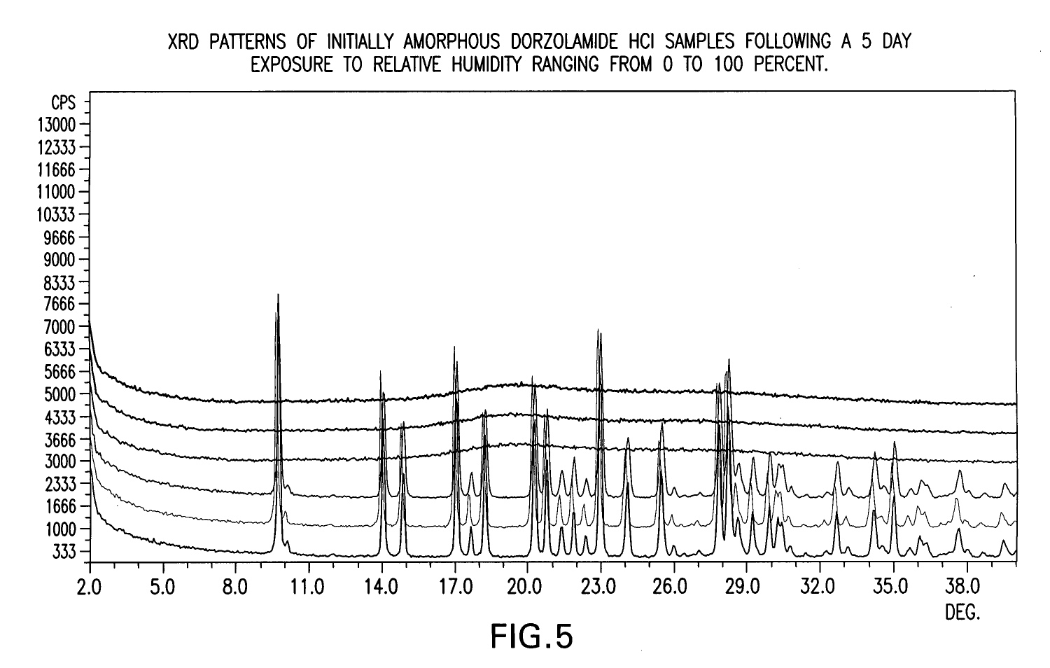

In another embodiment, the present invention provides a process for preparing Dorzolamide HCl Form II, comprising exposing amorphous Dorzolamide HCl at a temperature of more than about 25° C. to an elevated relative humidity of more than about 40 percent, for more than about 5 days, and recovering Dorzolamide HCl Form II. Preferably, the elevated relative humidity is at least about 60 percent, more preferably, at least about 80 percent, and, most preferably, about 100 percent. X ray diffraction patterns of initially amorphous Dorzolamide HCl samples following 5 day exposures over a relative humidity range of from 0 to 100 percent are depicted in FIG. 5. The relative humidity for the exposure of the top X-ray diffraction pattern in FIG. 5 is 0 percent, that for the second from the top is 20 percent, that for the third from the top is 40 percent, that for the fourth from the top is 60 percent, that for the fifth from the top is 80 percent, and that for the bottom X ray diffraction pattern in FIG. 5 is 100 percent.

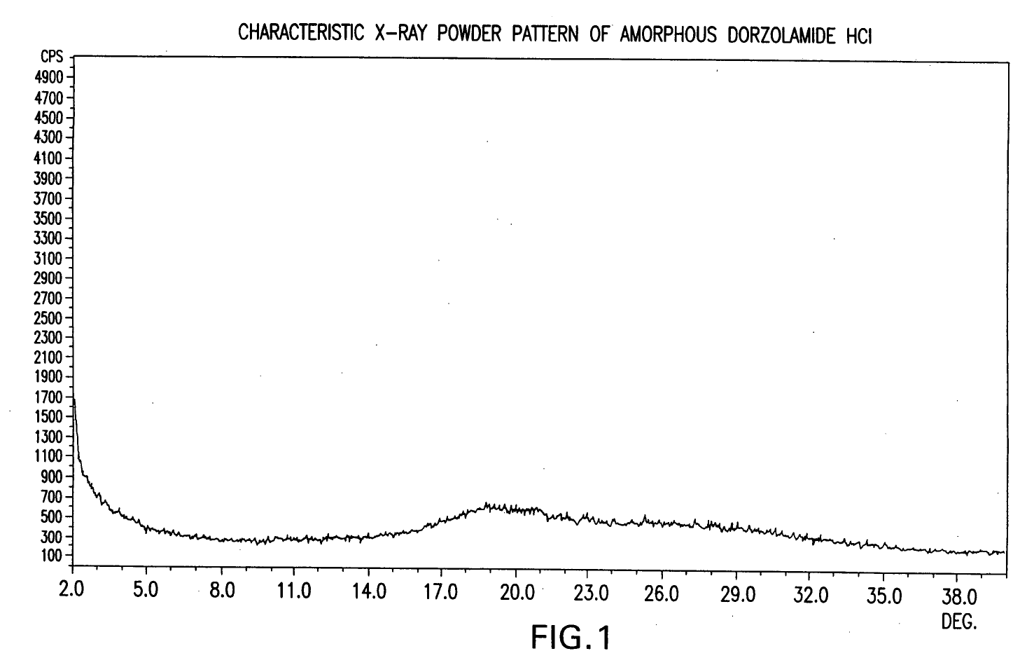

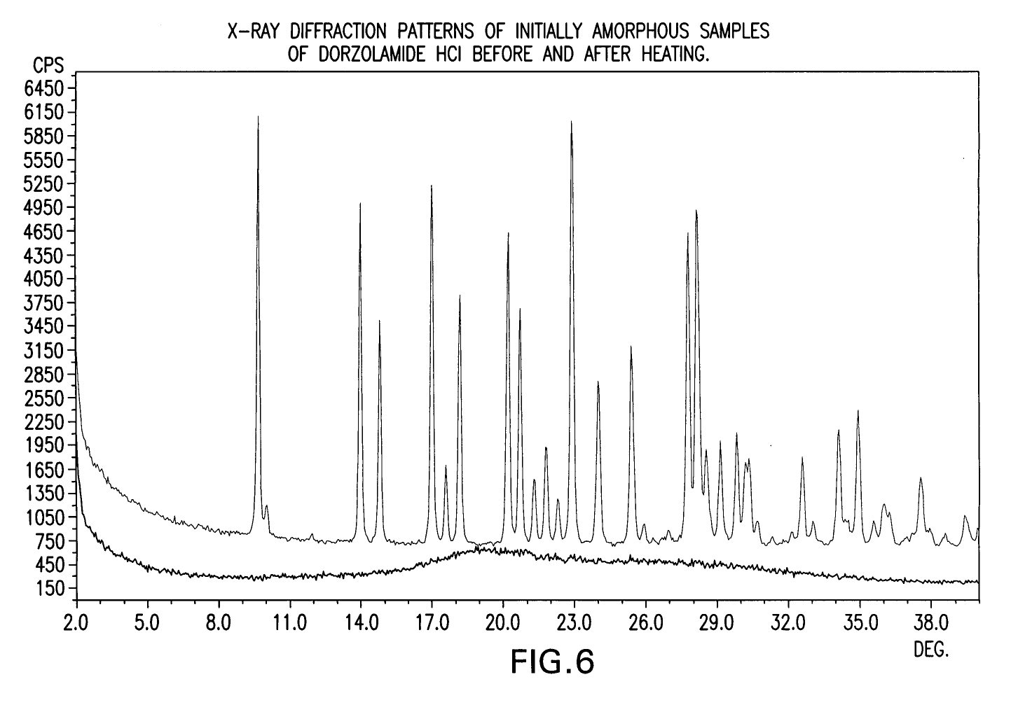

In a particular embodiment, the method of making Dorzolamide HCl Form II comprises heating a sample of amorphous Dorzolamide HCl, preferably to a temperature of about 160° C. for a period of time of about 15 minutes. X-ray diffraction patterns for an initial sample of amorphous Dorzolamide HCl before and after heating at 160° C. for 15 minutes are provided in FIG. 6. The bottom X-ray diffraction pattern in FIG. 6 is that of the sample before heating, and corresponds to that of amorphous Dorzolamide HCl, as depicted in FIG. 1. The top X-ray diffraction pattern in FIG. 6 is that of the sample after heating, is that of Dorzolanide HCl Form II.

In one embodiment, the present invention is directed to crystalline Dorzolamide hydrochloride acetamide solvate. Preferably, the Dorzolamide hydrochloride acetamide solvate contains about 1:1 ratio of dorzolamide hydrochloride and N,N-dimethylacetamide.

In one embodiment, the present invention is directed to a crystalline form of Dorzolamide hydrochloride, designated as Form IV characterized by at least one of an X-ray powder diffraction pattern, having peaks at about 8.5°, 11.4°, 15.9°, and 18.7°±0.2° 2θ, an FT-IR spectrum, having infrared peaks at about 3275, 1620, 1305 and 1144 cm⁻¹, and a DSC thermogram, having two endothermic peaks at about 174° C. and in the range of about 278° to about 281° C. The crystalline form may be further characterized by a X-ray powder diffraction pattern, having peaks at about 12.3°, 16.6°, 17.1°, 18.1°, 20.7°, and 21.0°±0.2° 2θ. The crystalline form may be further characterized by an FT-IR spectrum, having peaks at 2669, 1405, 1343, 1317, 1273, 1172, 1040, 1025, 988, 930, 782, 721 and 684 cm⁻¹. The crystalline form may be further characterized by a TGA thermogram with a small weight loss of about 0.2 percent up to about 130° C. that corresponds to a Karl Fischer titration. The TGA thermogram also indicates a sharp weight loss step of about 19.2 percent that corresponds to the theoretical Dorzolamide hydrochloride to N,N-dimethylacetamide solvate ratio of about 1:1. That is, Dorzolamide hydrochloride crystal Form IV is an N,N-dimethylacetamide solvate in a ratio of about 1:1. The TGA curve of Dorzolamide HCl Form IV also shows a sharp weight loss at about 280° C. Appropriate PXRD, FT-IR, DSC and TGA figures correspond to figure numbers 7, 8, 9 and 10.

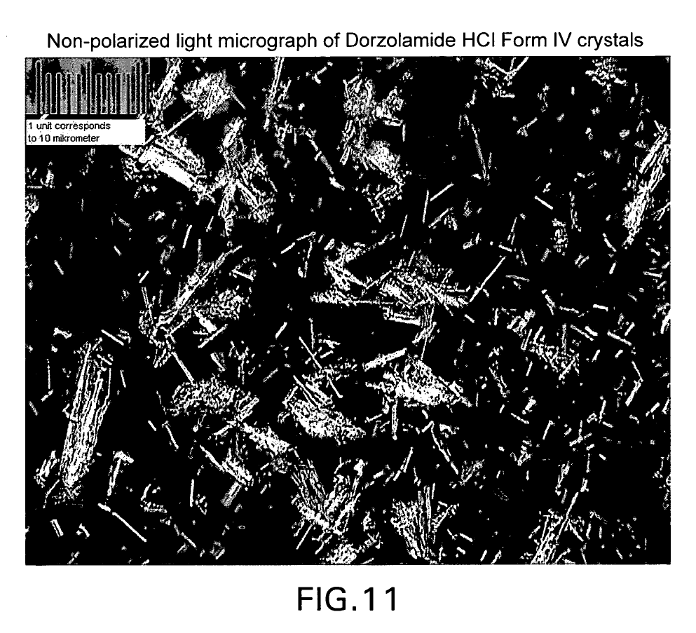

A microscopic analysis of Form IV of dorzolamide HCl determined that the crystals have a thin rod shape. The maximum crystal length observed was about 200 μm. The morphology and particle size of single crystals of Dorzolamide hydrochloride were analyzed using a polarizing light Microscope. A non-polarized light micrograph of particles of crystalline Dorzolamide HCl is provided in FIG. 11.

In another embodiment, the present invention provides a process for preparing Dorzolamide HCl Form IV by precipitating it from a solution comprising Dorzolamide hydrochloride and N,N-dimethylacetamide. The process comprises dissolving Dorzolamide hydrochloride in N,N-dimethylacetamide to obtain a solution, cooling the solution until a slurry is obtained, and recovering the crystalline form. The solution may further comprise an organic solvent selected from the group consisting of C₁-C₆ alcohols, C₂-C8 alkyl ketones and mixtures thereof. Preferably, the organic solvent is selected from the group consisting of ethanol, acetone, 1-propanol, 2-propanol, 1-butanol, 2-butanol and t-butanol. More preferably, the organic solvent is ethanol, acetone, 2-propanol, or a mixture thereof. Preferably, the solution is stirred. Preferably, the solution is maintained at a temperature of from about 550 to about 170° C., more preferably, at a temperature of from about 55° to about 65° C. The solution is maintained, preferably by stirring. Preferably, the solution is cooled to a temperature of about −25° C. to about 25° C. More preferably, the solution is cooled to a temperature of from about −10° C. to about 10° C. Preferably, the recovering step comprises filtering the slurry and drying it, preferably under vacuum, to give the Dorzolamide hydrochloride Form IV. Preferably, the Dorzolamide hydrochloride form IV obtained contains about 19 to about 19.8 weight percent, more preferably, 19.8 weight percent of acetamide solvate.

The present invention comprises pharmaceutical composition comprising Dorzolamide hydrochloride form IV or amorphous form and at least one pharmaceutically acceptable excipient.

The present invention comprises pharmaceutical composition comprising Dorzolamide hydrochloride of any of the forms II, IV or amorphous form made by the processes of the invention, and at least one pharmaceutically acceptable excipient.

The present invention further encompasses a process for preparing a pharmaceutical formulation comprising combining the Dorzolamide hydrochloride of any of the forms II, IV or amorphous form made by the processes of the invention, with at least one pharmaceutically acceptable excipient.

The present invention further encompasses a process for preparing a pharmaceutical formulation comprising combining the Dorzolamide hydrochloride form IV or amorphous form with at least one pharmaceutically acceptable excipient.

The present invention further encompasses the use of dorzolamide hydrochloride form IV or amorphous form for the manufacture of a pharmaceutical composition.

The present invention further encompasses the use of dorzolamide hydrochloride of any of the forms II, IV or amorphous form made by the processes of the invention, for the manufacture of a pharmaceutical composition.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of Dorzolamide hydrochloride and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdon®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sole, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the Dorzolamide hydrochloride of any of the forms II, IV or amorphous described herein and any other solid ingredients are dissolved or suspended in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of Dorzolamide hydrochloride of any of the forms II, IV or amorphous contained in a pharmaceutical composition for reducing cholesterol according to the present invention is not specifically restricted; however, the dose should be sufficient to treat, ameliorate, or reduce the condition.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### Experimental Methodology

An ARL X-ray powder diffractometer model X TRA-030 with a Peltier detector and a round, standard aluminum sample holder with round zero background quartz plate is preferably used for XRD analysis. The scanning parameters are as follows: the range is preferably about 2° to about 40° 2θ over a substantially continuous scan at a rate of about 3° per minute. The accuracy of peak positions is defined as ±0.2° due to experimental differences, such as instrumentations, sample preparations, and the like.

FT-IR spectrograms of samples of the amorphous form of Dorzolamide hydrochloride of the invention are preferably obtained with a Perkin-Elmer Spectrum 1000 Spectrometer at a resolution of preferably about 4 cm⁻¹ with about 16 scans, in the range of about 4000 to about 400 cm⁻¹. Samples are analyzed preferably in a Nujol mull, using an empty cell as a background when spectra are recorded.

Differential Scanning Calorimetry (DSC) measurements are taken preferably on a Mettler Toledo DSC 822^{e}/700. Sample weights are preferably in the range of about 3 to about 5 mg with a heating rate of about 10° C. per minute. The number of holes in the crucible used is preferably 3, the flow rate of the nitrogen (N2) stream is preferably about 40 ml per minute, the scan range is preferably about 30° to about 250° C., and the heating rate is preferably about 10° C. per minute.

Thermal Gravimetric Analysis (TGA) is preferably performed with a Mettler Toledo TGA/SDTA 851^{e}. Sample weights preferably range from about 7 to about 15 mg, the heating rate is preferably about 10° C. per minute in a N₂ stream having a flow rate of about 50 ml per minute over a scan range of about 30° to about 250° C.

The morphology and particle size of single crystals is preferably analyzed using a polarizing light Microscope, with parallel Nicols.

Spray drying was performed on a Buchi Mini Spray dryer B-290. The nozzle diameter was 0.7 mm.

### EXAMPLES

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### Example 1

### Preparation of Amorphous Dorzolamide Hydrochloride

A 15 g sample of Dorzolamide hydrochloride solid is dissolved in 300 ml water. After stirring for 15 minutes, the solution is filtered, and then diluted with 100 ml of water, providing a 7.5 m/m percent dorzolamide solution. The resulting solution is fed into a Buichi Spray Dryer Type 191, and dried using the following drying parameters: an inlet temperature of about 120° to about 125° C., an outlet temperature of about 53° to about 60° C., an air speed of about 600 liters per hour, a feed rate of about 225 to about 375 ml per hour for about 90 minutes. The drying process produces 7.8 g of dried material that is received in a cyclon collector. An XRD analysis demonstrates that the product is the amorphous form of Dorzolamide hydrochloride.

### Example 2

### Effect of Varying Relative Humidity on Amorphous Dorzolamide Hydrochloride

Each of a series of 200 mg samples of amorphous Dorzolamide hydrochloride are exposed to a relative humidity (RH) of one of 0, 20, 40, 60, 80, and 100 percent for a period of 5 days at room temperature in a hygroscopicity chamber. The loss on drying (LOD) of the samples is measured by TGA before and after exposure, and the values obtained are plotted against the RH values. In addition, the crystal form of each sample is measured by XRD before and after the exposure. The results are summarized in Table 1, and the equilibrium water content of the samples of Dorzolamide HCl at different values of the relative humidity are also provided.

The results are supported by the XRD patterns illustrated in FIG. 5, that show that the Dorzolamide HCl is amorphous at a relative humidity of 0 percent, 20 percent, and 40 percent, and transforms to crystal form II at 60 percent, 80 percent, and 100 percent RH.

### Example 3

### Preparation of Form II of Dorzolamide Hydrochloride

Several 200 mg samples of amorphous Dorzolamide HCl were exposed to a temperature of about 160° C. for about 15 minutes in an oven in an open glass weighing bottle, placed in a desiccator, and cooled to room temperature. After cooling, the samples were analyzed by XRD. Exposure of the amorphous form of the Dorzolamide HCl changed the material to the crystalline, anhydrous form II.

### Example 4

### Preparation of Form IV of Dorzolamide Hydrochloride

Two grams of Dorzolamide hydrochloride and 4 ml of N,N-dimethylacetamide were stirred together at reflux temperature. The resulting solution was then cooled to room temperature, and aged for 6 hours, forming a slurry. The slurry was filtered and dried at 50° C. under vacuum to give 1.56 g of Dorzolamide hydrochloride Form IV.

### Example 5

### Preparation of Form IV of Dorzolamide Hydrochloride

Two grams of Dorzolamide hydrochloride and 10 ml of N,N-dimethylacetamide were stirred together at 60° C., and 20 ml of dry ethanol was added to the resulting solution. The solution was cooled to −20° C., and aged for 20 hours, forming a slurry. The slurry was filtered and dried at 50° C. under vacuum to give 1.82 g of Dorzolamide hydrochloride Form IV.

### Example 6

### Preparation of Form IV of Dorzolamide Hydrochloride

Two grams of Dorzolamide hydrochloride and 10 ml of N,N-dimethylacetamide were stirred together at 60° C., and 40 ml of dry ethanol was added to the resulting solution. The solution was cooled to −20° C., and aged for 20 hours, forming a slurry. The slurry was filtered and dried at 50° C. under vacuum to give 1.90 g Dorzolamide hydrochloride Form IV.

### Example 7

### Preparation of Form IV of Dorzolamide Hydrochloride

Two grams of Dorzolamide hydrochloride and 10 ml of N,N-dimethylacetamide were stirred together at 60° C., and 40 ml of acetone were added to the resulting solution. The solution was cooled to room temperature, and aged for 10 hours, forming a slurry. The slurry was filtered and dried at 50° C. under vacuum to give 2.09 g Dorzolamide hydrochloride Form IV.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

What is claimed:

1. An amorphous form of Dorzolamide hydrochloride.

2. The amorphous form of claim 1, having a PXRD pattern substantially as depicted in FIG. 1.

3. The amorphous form of claim 1, having a DSC thermogram with exothermic peak at about 130° C. to about 180° C. and one endothermic peak at about 284° C.

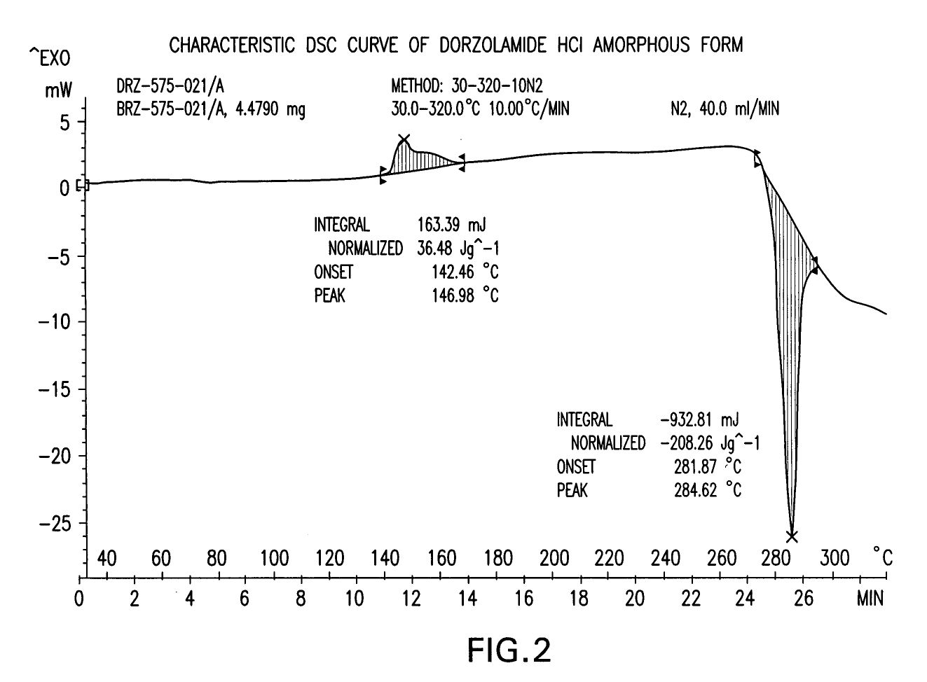

4. The amorphous form of claim 3, having a DSC thermogram substantially as depicted in FIG. 2.

5. The amorphous form of claim 1, having a TGA showing a weight loss of about of about 3 percent up to 170° C. and a sharp weight loss at about 230° C.

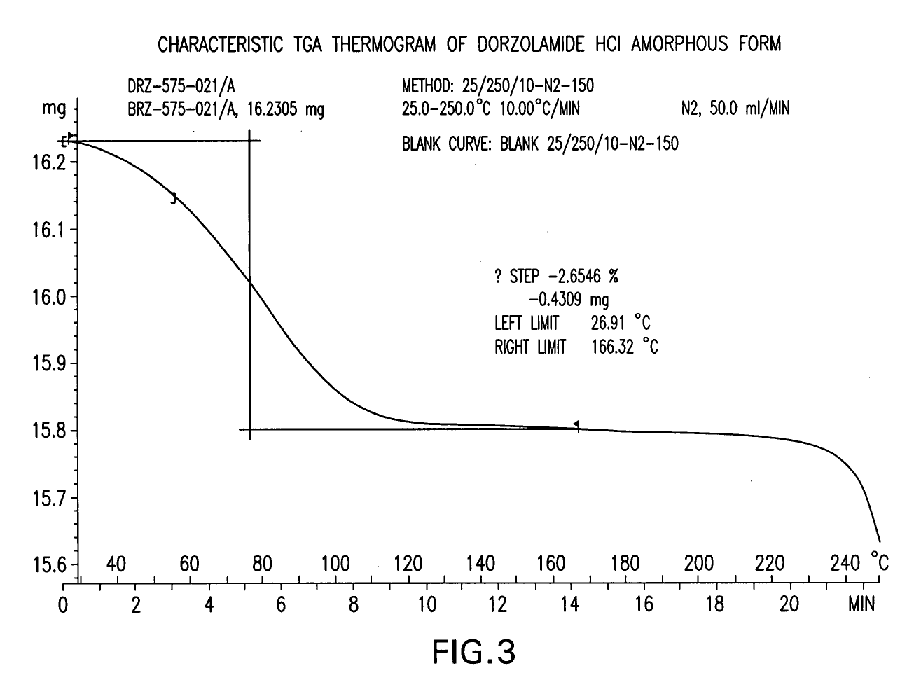

6. The crystalline form of claim 5, having a TGA thermogram substantially as depicted in FIG. 3.

7. The amorphous form of claim 1, having irregular morphology.

8. The amorphous form of claim 7, having irregular morphology with particles having a size of up to about 10 to about 20 μm.

9. The amorphous form of claim 7, having a non-polarized light micrograph of particles of Dorzolamide HCl substantially as depicted in FIG. 4.

10. The amorphous form of claim 1, containing less than about 10% of any crystalline form of dorzolamide hydrochloride.

11. The amorphous form of claim 10, containing less than about 5% of any crystalline form of dorzolamide hydrochloride.

12. The amorphous form of claim 11, containing less than about 2% of any crystalline form of dorzolamide hydrochloride.

13. The amorphous form of claim 12, containing less than about 1% of any crystalline form of dorzolamide hydrochloride.

14. The amorphous form of claim 13, containing less than about 0.2% of any crystalline form of dorzolamide hydrochloride.

15. An amorphous form of dorzolamide that does not spontaneously convert to any crystalline form, at a temperature of about 40° C. or less, at a relative humidity of less than about 40 percent, for a period of time of at least about 3 months.

16. A process for preparing amorphous form of dorzolamide hydrochloride of claim 1, by spray-drying a solution of Dorzolamide hydrochloride in a solvent selected from the group consisting of water, methanol, ethanol and mixtures thereof.

17. The process of claim 16, wherein the solution has a Dorzolamide hydrochloride concentration is of about 10 to about 100 g/l.

18. The process of claim 16, wherein the solution is stirred for about 10 to about 60 minutes.

19. The process of claim 16, wherein the solution is filtered.

20. The process of claim 19, wherein the filtered solution is diluted with water to provide a dorzolamide solution having a concentration of from about 0.5 to about 10 m/m percent.

21. The process of claim 16, wherein the solution is spray dried at an inlet temperature of about 110° C. to about 130° C.

22. The process of claim 21, wherein the solution is spray dried at an inlet temperature of about 120° C. to about 125° C.

23. The process of claim 16, wherein the solution is spray dried at an outlet temperature of about 40° C. to about 60° C.

24. The process of claim 23, wherein the solution is spray dried at an outlet temperature of about 53° to about 60° C.

25. The process of claim 16, wherein the air speed during the spray-drying is of about 600 liters per hour.

26. A process for preparing Dorzolamide HCl Form II comprising exposing amorphous Dorzolamide HCl to a temperature of more than about 25° C. to an elevated relative humidity of more than about 40 percent, for more than about 5 days, and recovering Dorzolamide HCl Form II.

27. The process of claim 26, wherein the elevated relative humidity is of at least about 60 percent.

28. The process of claim 27, wherein the elevated relative humidity is of at least about 80 percent.

29. The process of claim 28, wherein the elevated relative humidity is of at least about 100 percent.

30. Crystalline Dorzolamide hydrochloride acetamide solvate.

31. The crystalline Dorzolamide hydrochloride acetamide solvate of claim 30, containing about a 1:1 ratio of dorzolamide hydrochloride and N,N-dimethylacetamide.

32. A crystalline form of Dorzolamide hydrochloride characterized by at least one of an X-ray powder diffraction pattern, having peaks at about 8.5°, 11.4°, 15.9° and 18.7°±0.2° 2θ, an FT-IR spectrum, having peaks at about 3275, 1620, 1305 and 1144 cm⁻¹, and a DSC thermogram, having two endothermic peaks at about 174° C. and in the range of about 278° to about 281° C.

33. The crystalline form of claim 32, characterized by an X-ray powder diffraction pattern, having peaks at about 8.5°, 11.4°, 15.9° and 18.7°±0.2° 2θ.

34. The crystalline form of claim 33, wherein the crystalline form is further characterized by a X-ray powder diffraction pattern, having peaks at about 12.3°, 16.6°, 17.1°, 18.1°, 20.7°, and 21.0°±0.2° 2θ.

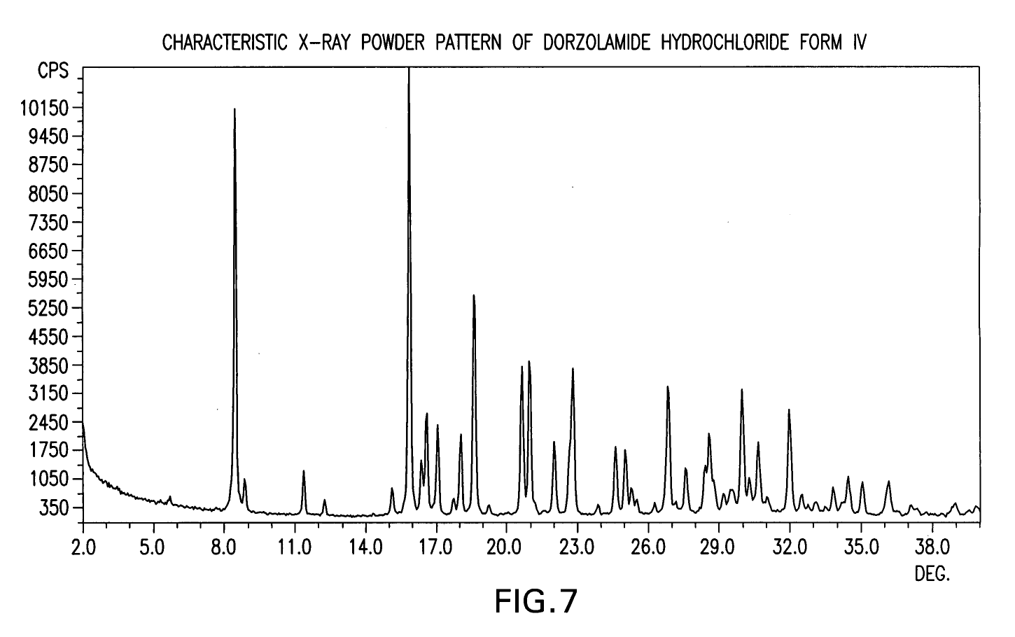

35. The crystalline form of claim 34, having an XRD pattern substantially as depicted in FIG. 7.

36. The crystalline form of claim 32, characterized by an FT-IR spectrum, having peaks at about 3275, 1620, 1305 and 1144 cm⁻¹.

37. The crystalline form of claim 36, wherein the crystalline form is further characterized by an FT-IR spectrum, having peaks at about 2669, 1405, 1343, 1317, 1273, 1172, 1040, 1025, 988, 930, 782, 721 and 684 cm⁻¹.

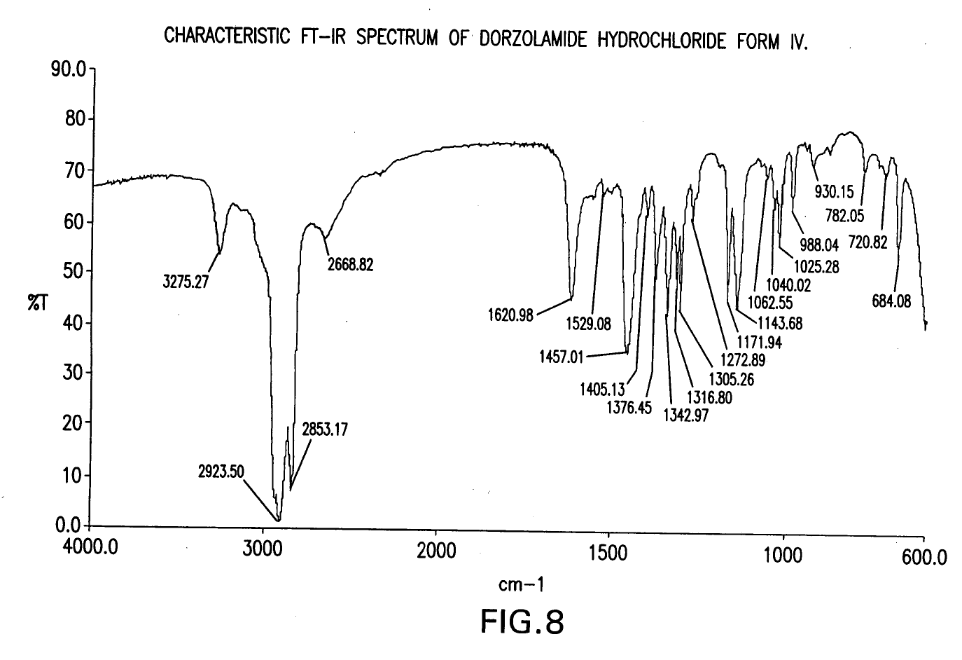

38. The crystalline form of claim 37, having an FT-IR spectrum substantially as depicted in FIG. 8.

39. The crystalline form of claim 32, characterized by DSC thermogram with two endothermic peaks at about 174° C. and in the range of about 278° to about 281° C.

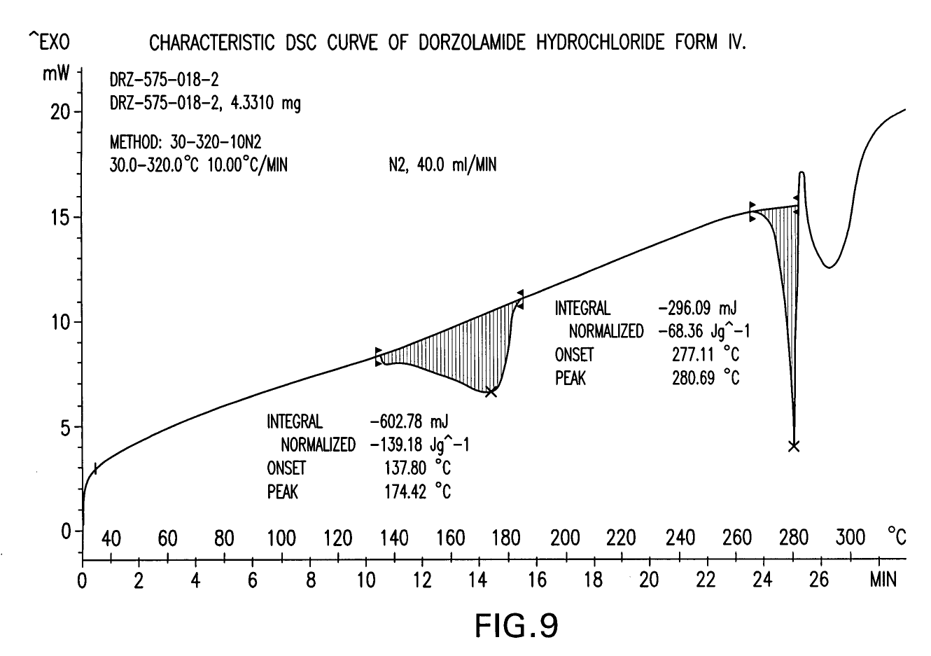

40. The crystalline form of claim 39, having a DSC thermogram substantially as depicted in FIG. 9.

41. The crystalline form of claim 32, wherein the crystalline form is an acetamide solvate.

42. The crystalline form of claim 41, containing about a 1:1 ratio of dorzolamide hydrochloride and N,N-dimethylacetamide.

43. The crystalline form of claim 32, wherein the crystalline form is further characterized by a TGA thermogram with a small weight loss of about 0.2 percent up to about 130° C.

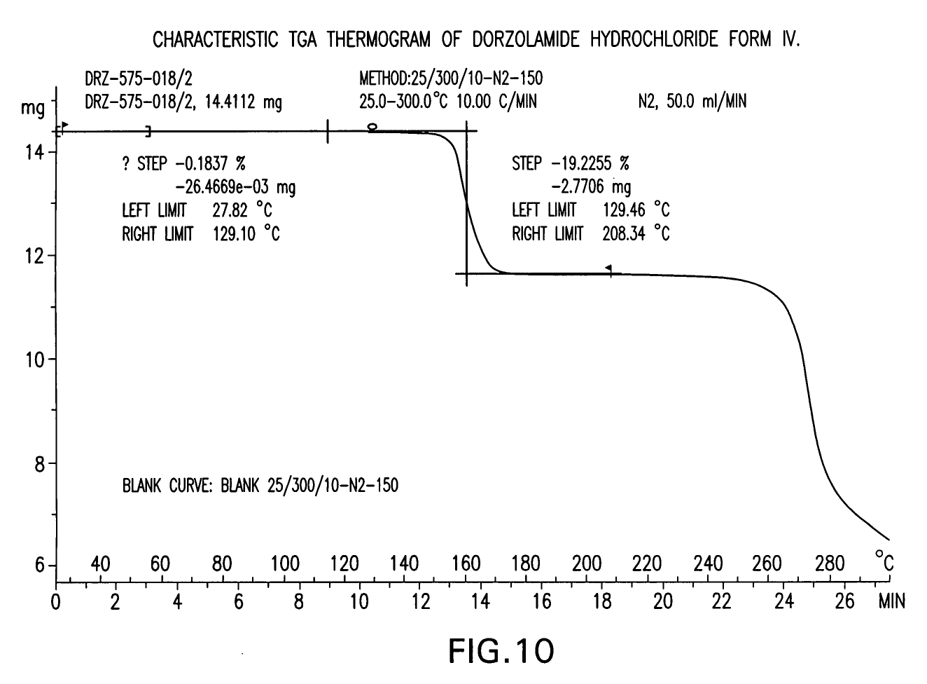

44. The crystalline form of claim 43, having a TGA thermogram substantially as depicted in FIG. 10.

45. The crystalline form of claim 32, having a maximum crystal length of about 200 μm.

46. The crystalline form of claim 45, having a non-polarized light micrograph of particles of Dorzolamide HCl substantially as depicted in FIG. 11.

47. A process for preparing the crystalline form of Dorzolamide HCl of claim 32 comprising, dissolving Dorzolamide hydrochloride in N,N-dimethylacetamide to obtain a solution, cooling the solution until a slurry is obtained, and recovering the crystalline form.

48. The process of claim 47, wherein the solution further comprises an organic solvent selected from the group consisting of C₁-C₆ alcohols, C₂-C₈ alkyl ketones and mixtures thereof.

49. The process of claim 48, wherein the organic solvent is selected from the group consisting of ethanol, acetone, 1-propanol, 2-propanol, 1-butanol, 2-butanol and t-butanol.

50. The process of claim 49, wherein the organic solvent is ethanol, acetone, 2-propanol, or a mixture thereof.

51. The process of claim 47, wherein the solution maintained at a temperature of from about 55° to about 170° C.

52. The process of claim 51, wherein the solution maintained at a temperature of from about 55° to about 65° C.

53. The process of claim 47, wherein the solution is cooled to a temperature of from about −25° to about 25°.

54. The process of claim 53, wherein the solution is cooled to a temperature of from about −10° to about 10°.

55. The process of claim 47, wherein the recovering step comprises filtering the slurry and drying it.

56. The process of claim 47, wherein the recovered Dorzolamide hydrochloride form contains about 19 to about 19.8 weight percent of acetamide solvate.

57. The process of claim 56, wherein the recovered Dorzolamide hydrochloride form contains about 19.8 weight percent of acetamide solvate.

58. A pharmaceutical composition, comprising the Dorzolamide hydrochloride of any one of claims 1 and 32 and at least one pharmaceutically acceptable excipient.

59. A process for preparing a pharmaceutical formulation comprising combining the Dorzolamide hydrochloride of any one of claims 1 and 32, with at least one pharmaceutically acceptable excipient.

60. Use of dorzolamide hydrochloride of any one of claims 1 and 32 for the manufacture of a pharmaceutical composition.

61. A pharmaceutical composition comprising the Dorzolamide hydrochloride made by the processes of any one of claims 16, 26 and 47 and at least one pharmaceutically acceptable excipient.

62. A process for preparing a pharmaceutical formulation comprising combining the Dorzolamide hydrochloride made by the processes of any one of claims 16, 26 and 47, with at least one pharmaceutically acceptable excipient.

63. Use of dorzolamide hydrochloride made by the processes of any one of claims 16, 26 and 47 for the manufacture of a pharmaceutical composition.
